(19) United States
(12) Patent Application Publication
(10) Pub. No.: US 2025/0332737 A1
(43) Pub. Date: Oct. 30, 2025

(54) ADAPTIVE DAMPER FOR COMPUTER-ASSISTED SYSTEM

(71) Applicant: Intuitive Surgical Operations, Inc., Sunnyvale, CA (US)

(72) Inventor: J. Scot Hart, San Carlos, CA (US)

(73) Assignee: Intuitive Surgical Operations, Inc., Sunnyvale, CA (US)

(21) Appl. No.: 18/869,918

(22) PCT Filed: May 30, 2023
(86) PCT No.: PCT/US2023/023797

§ 371 Date: Nov. 27, 2024

Related U.S. Application Data
(60) Provisional application No. 63/347,304, filed on May 31, 2022.

(51) Int. Cl. B25J 13/02; A61B 34/30; A61B 34/35; B25J 9/16

(52) U.S. Cl. CPC B25J 13/025; B25J 9/1689; A61B 2034/301; A61B 34/35

(57) ABSTRACT

A computer-assisted system may include a manipulator arm configured to support an instrument, an input device configured to accept user commands to move the instrument, and a damping system coupled to the input device. A controller of the computer assisted system may be configured to determine an instrument power metric and an input device power metric. The controller may also determine a damping to be applied to the input device based on the instrument power metric and the input device power metric and cause the damping system to adjust the damping applied to the input device.

### CROSS-REFERENCE TO RELATED APPLICATIONS

This application claims the benefit of priority under 35 U S.C. § 119(e) to U.S. Provisional Patent Application Ser. No. 63/347,304, filed on May 31, 2022, which is hereby incorporated by reference herein in its entirety.

### FIELD

Disclosed embodiments are related to adaptive dampers for computer-assisted systems and related methods of use.

### BACKGROUND

Computer-assisted electronic systems are being used more and more often. This is especially true in industrial, entertainment, educational, and other settings. As a medical example, the medical facilities of today have large arrays of electronic devices being found in operating rooms, interventional suites, intensive care wards, emergency rooms, and/or the like. Many of these electronic devices may be capable of autonomous or semi-autonomous motion. Personnel may teleoperatively control the motion and/or operation of follower electronic devices using one or more leader input devices located at a user control system. As an example, robotic telemedical systems permit medical personnel to teleoperate instruments to perform medical procedures from beside the patient or from remote locations. As a specific example, minimally invasive, robotic telesurgical systems permit surgeons to operate on patients from bedside or remote locations. Telesurgery refers generally to surgery performed using surgical systems where the surgeon uses some form of remote control, such as a servomechanism, to manipulate surgical instrument movements rather than directly holding and moving the instruments by hand. In some systems with actuator systems coupled to the input devices, the actuator systems may be driven to provide tactile feedback through the input devices. Thus, improved apparatus and method for the operation of computer-assisted electronic systems are desired.

### SUMMARY

In some embodiments, a computer-assisted system comprises a manipulator arm configured to support an instrument, an input device configured to accept user commands to move the instrument, a damping system coupled to the input device, and a controller comprising at least one processor. The controller is configured to determine an instrument power metric indicative of an amount of power of an instrument interaction, the instrument interaction comprising a physical interaction between at least a portion of the instrument and an instrument environment containing the instrument, determine an input device power metric indicative of an amount of power of a feedback provided by the input device in response to the instrument interactiondetermine a damping to be applied to the input device based on the instrument power metric and the input device power metric, and cause the damping system to apply the damping to the input device.

In some embodiments, a method of controlling a computer-assisted system configured to support an instrument, the computer-assisted system comprising an input device configured to control movement of the instrument and a damping system configured to apply force to the input device, comprises determining a component of a change in force at the instrument, the component correlating with a direction of motion of the input device, and, in response to a determination that an environmental stiffness experienced by the instrument exceeds a threshold stiffness, determining a feedback force based at least in part on the environmental stiffness and the component of the change in force. The method further comprises causing the damping system to drive the input device to apply the feedback force. In some embodiments, a non-transitory computer-readable storage medium may store instructions that, when executed by at least one processor associated with a computer-assisted device, causes the at least one processor to perform the method.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1 is a diagram of a computer-assisted system according to some embodiments;

FIG. 2 is block diagram of a computer-assisted system according to some embodiments;

FIG. 3 is a diagram of a control loop for a computer-assisted system according to some embodiments;

FIG. 4 is a simplified diagram of a computer-assisted system during operation according to some embodiments;

FIG. 5 is a simplified diagram of an input device of a computer-assisted system according to some embodiments;

FIG. 6 is a simplified diagram of a computer-assisted system during operation according to some embodiments;

FIG. 7 depicts graphs of experimental results of a computer-assisted system according to some exemplary embodiments described herein; and

FIG. 8 depicts a flow chart for a method of operating a computer-assisted system according to some embodiments.

### DETAILED DESCRIPTION

Some computer-assisted systems comprise follower devices that are teleoperated in response to user input received at input devices. Where such teleoperation is performed with input devices coupled with actuator systems, the actuator systems may be driven to provide tactile feedback through the input devices based on forces determined to be experienced by some part of the follower device, or by a component supported by the follower devices. Controlling such actuator systems to provide such tactile feedback can be difficult, or can introduce unfavorable or inaccurate tactile feedback to a user of the computer-assisted system. For example, in a computer-assisted system where an input device is employed to teleoperate a manipulator arm, phase loss may be introduced between the input device and the manipulator arm due to physical separation of the input device from the manipulator arm and the associated time delay in transmitting information. In some cases where a computer-assisted system is used to facilitate teleoperation of the manipulator arm, the phase loss may be significant and tend to generate instabilities in control of actuators of the input device and/or the manipulator arm. As another example, instabilities in the control of an actuator system used to provide user feedback through an input device based on interactions experienced by a follower manipulator arm of a computer-assisted system may also be caused by a change in operational environment for the manipulator arm. For example, a manipulator arm may support an instrument that, in some operating environments and instances, can engage with material such as objects that differ in rigidity or compliance. For example, the instrument may interact with one or more harder, or more rigid surfaces of an object as well as one or more softer (e.g., less hard), or more compliant (e.g., less rigid) surfaces of an object. As another example, the instrument may interact with harder, or more rigid bulk of an object in an environment as well as softer (e.g., less hard), or more compliant (e.g., less rigid) bulk of an object. As yet a further example, the instrument may interact with material in the environment that is not solid, such as liquid or gel-type materials (e.g., a bulk). As used herein, the hardness of an object may refer to an object's resistance to surface deformation and rigidity may be a measure of an object's or other material's resistance to bulk deformations. The change in the encountered environmental rigidity or compliance as the instrument interacts with these different surfaces, or bulks, or non-solid materials may also introduce instabilities in controlling actuators of an input device to provide feedback to the user due a rapid increase in force at the instrument.

The technique described in this disclosure can provide a variety of benefits in a computer-assisted system including an input device and a manipulator arm controlled by the input device, where the interaction of the manipulator arm (or a component supported by the manipulator arm, such as an instrument) is used to provide tactile feedback to a user of the input device. Example benefits include helping to retain stability in controlling the feedback provided by the input device, helping to improve or retain tracking accuracy between the commands received at the input device and the motions performed by the manipulator arm. In some embodiments, the disclosed technique reduces the amount of damping applied to an input device with, and applies little or no damping (e.g., barely or not detectable based on human senses) while a manipulator arm teleoperated by the input device is not experiencing reaction forces from the operating environment (e.g. the manipulator arm or an instrument supported by the manipulator arm is not in contact with any materials other than air or other gases, or in free space).

In some embodiments, the disclosed technique provides a controller configured to compensate for operating environment impedance encountered by a teleoperated manipulator arm with reduced likelihood of increasing a control loop gain drastically, which reduces the likelihood of unstable force feedback rendered at the input device. A potential benefit includes an adaptive controller for a computer-assisted system that enhances stable force feedback for an input device, by applying different damping to feedback provided by the input device when a manipulator arm is experiencing higher environmental stiffnesses (e.g. in contact with a more rigid object or harder surface of an object, etc.) than when the manipulator arm is experiencing lower environmental stiffnesses (e.g. in free space, in contact with more pliable material, etc.). For example, contact with a substantially rigid surface (e.g., bone or another instrument in a surgical example) or substantially rigid bulk, or some other material of greater rigidity (e.g., a more viscous fluid, a stiffer gel, etc.) may cause a rapid increase in control loop gain if not compensated for because of a rapid increase in input force measured at the instrument for a given amount of motion. As another example, the instrument may interact with harder, more rigid bulk of an object in an environment as well as softer (e.g., less hard), more compliant (e.g., less rigid) bulk of an object. As yet a further example, the instrument may interact with material in the environment that is not solid, such as liquid or gel-type materials. As used herein the hardness of an object may refer to an object's resistance to surface deformation and rigidity may be a measure of an object's or other material's resistance to bulk deformations. The change in the encountered environmental rigidity or compliance as the instrument interacts with these different surfaces, or bulks, or non-solid materials may also introduce instabilities in controlling actuators of an input device to provide feedback to the user due a rapid change in force at the instrument for small motions.

In some embodiments, the disclosed technique provides a controller configured to compensate for changes in the structural stiffness of the kinematic chain that comprises a teleoperated manipulator arm. For example, an extended manipulator arm may be more compliant and less stiff than a folded or retracted manipulator arm. Compensating for increased compliance and decreased stiffness may also result in increases in control loop gain if not compensated for. An extended manipulator arm generally will have a different bending stiffness because of its physical configuration compared to a folded or retracted manipulator arm, such that a force applied at the tip of an extended manipulator arm (versus a folded or retracted manipulator arm) is more able to flex the manipulator arm due to an increased lever arm.

In some embodiments, the disclosed technique allows the use of lower-mass input devices. And, in some embodiments, low-mass input devices may be associated with increased control loop gain, which can make the input device more susceptible to instability. The technique described herein can also be applied in a controller configured to compensate for increases in feedback control loop gain, for input device feedback forces caused by one or more sources.

In some embodiments, the computer assisted system may employ a method in which a power metric indicative of an amount of power of a physical interaction between the manipulator arm and its respective environment is determined. Likewise, a power metric based on the feedback force applied to an input device may be determined. Based on the determined power metrics for the input device and the manipulator arm, a damping of the input device may be determined and applied to the input device. In this manner, the damping of the input device may adjust automatically based on the feedback force applied to the input device and the physical interaction between the manipulator arm and its environment. The adaptive controller according to exemplary embodiments described herein may be employed in any computer-assisted robotic system with any associated instrument, including industrial computer-assisted systems, medical computer assisted systems, and other appropriate systems.

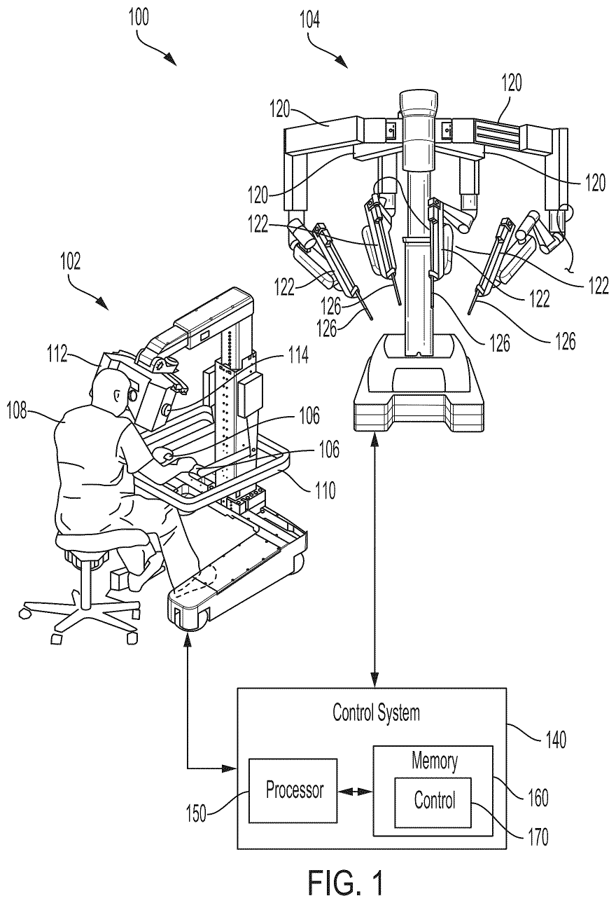

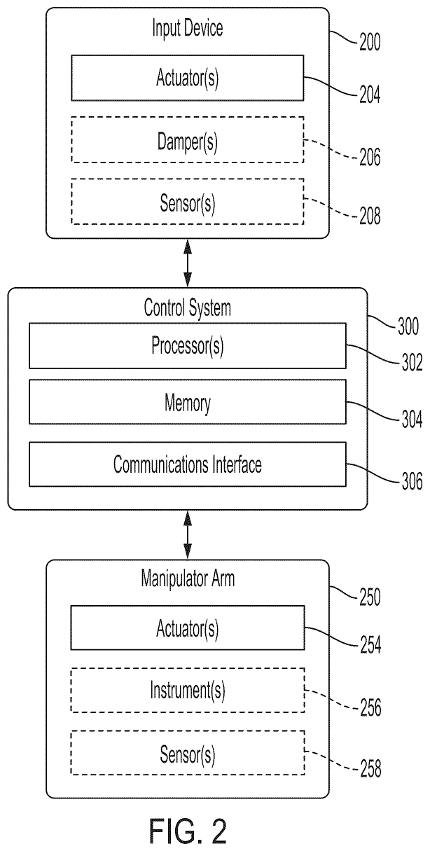

In some embodiments, a computer-assisted system includes a manipulator arm configured to support an instrument and an input device configured to accept user commands to move the instrument. The manipulator arm may be configured to support a plurality of instruments that may interact with one or more surfaces, bulks, or other materials. The computer-assisted system may also include an actuator system configured to apply feedback forces to the input device based on forces sensed at the manipulator arm. For example, in the case of a tele-operated computer-assisted system, the actuator system may be driven to apply forces to the input device to mirror or represent forces sensed or detected at a manipulator arm located remotely from the input device. In this manner, the actuator system may be employed to provide force-feedback to a user of the input device. The computer-assisted system may also include a controller configured to control forces applied to the input device, where the controller includes at least one processor configured to execute programming instructions which cause the at least one processor to perform exemplary methods described herein. Exemplary computer-assisted systems are discussed further with reference to at least FIGS. 1-2.

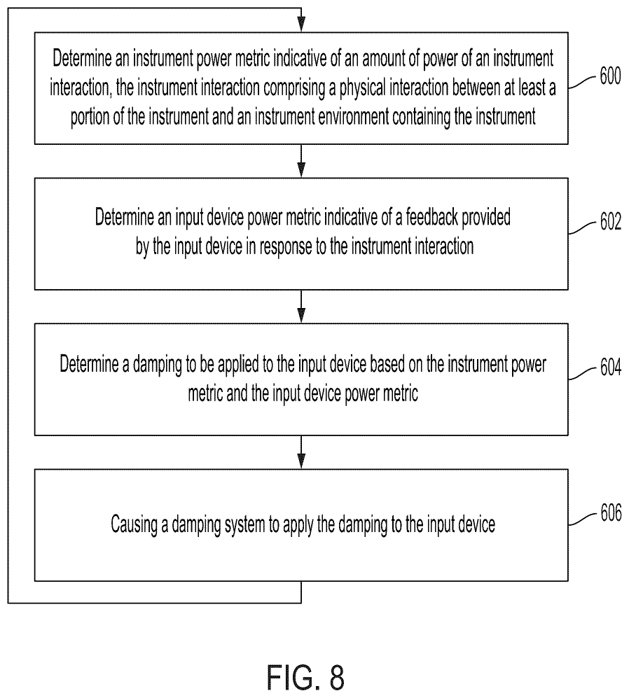

In some embodiments, a method of controlling a computer-assisted system including a manipulator arm and an input device includes measuring or estimating power flowing into an instrument supported by the manipulator arm from the environment surrounding the instrument (e.g., a power metric). For example, power may be measured or estimated based on contact between the instrument and a work piece or contact between the instrument and another instrument. Measuring or estimating power flowing into the instrument may include measuring forces at the instrument or elsewhere on the manipulator arm as well as measuring or determining a velocity of the instrument. The method may also include measuring or estimating power flowing into the user input device by measuring or determining the feedback forces applied to the input device as well as measuring or determining a velocity of the input device. The method may also include comparing (after optional scaling) the power metrics for the manipulator arm and the input device. In some embodiments, the comparison may be a difference. Based on the comparison between the manipulator arm power metric and the input device power metric, a damping for the input device is determined. In some embodiment, such a determination is based primarily on whether the manipulator arm power metric exceeds the input device power metric. In the case the manipulator arm power metric is greater (e.g., for a first range of differences), greater damping may be applied to the input device, for example, based on a strictly monotonic relationship (e.g., a positive monotonic relationship) between the damping and the difference between the manipular arm power metric and input device power metric. In the case the manipulator arm power metric is less than or equal to the input device power metric (e.g., for a second range of differences), the damping may be set to a baseline value (e.g., zero or a non-zero amount). This baseline value may not be set until the input device power metric exceeds or equals the manipulator arm power metric, so as to not introduce instabilities in the input device. In this manner, the damping is driven based on a difference in comparatively scaled power metrics for the input device and the manipulator arm. An exemplary method of controlling a computer-assisted system is discussed further with reference to exemplary FIG. 8.

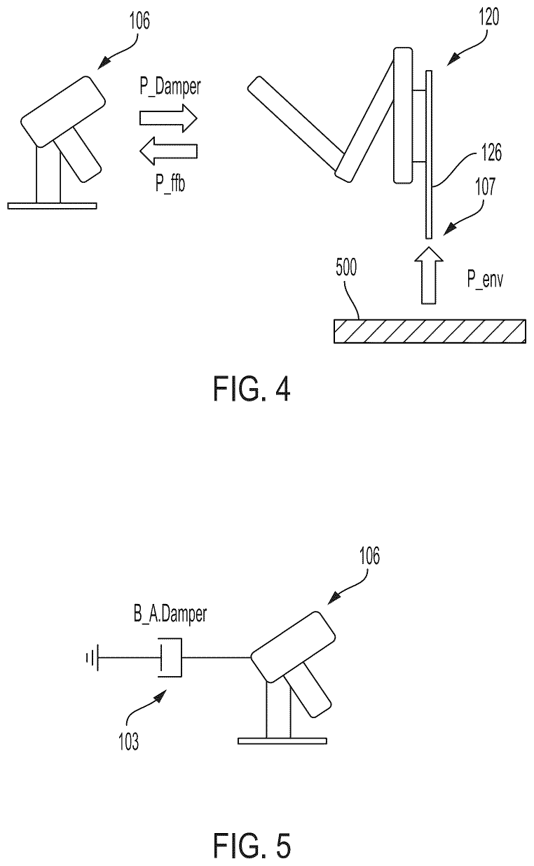

According to exemplary embodiments described herein, power metrics may be representative of power over a period of time (e.g., energy). For example, in some embodiments, a manipulator arm power metric may be indicative of an energy of instrument interaction with an instrument environment. Likewise, an input device power metric may be indicative of a feedback provided by the input device in response to the instrument interaction. A power metric may comprise an accumulation of power over a follower time period (e.g., a passage of time). The follower time period may begin at a moment where an input device is used and feedback force is provided at the user input device, and may end until the input device is released and feedback force is no longer rendered at the user input device. In some embodiments, the follower time period may be any length of time during which the input device is used by a user. In some embodiments, the follower time period may be between 1 ms and 7 hours. According to such embodiments, the power metric may be representative of energy of a manipulator arm interaction and an input device interaction over the follower time period. In some embodiments, a follower time period may be composed of several integrations of instantaneous power over an interval time period. That is, an overall integral of the instantaneous power may be determined by a summation of multiple interval integrations over the interval time periods, in some embodiments. For example, determining a power metric may comprise repeatedly integrating instantaneous power over the interval time period and summing each of the repeated interval integrations. In some embodiments, the interval time period may be at least partially determined by an interval of a control loop or servo cycle for the computer-assisted system. For example, the interval time period may be between 0.0005 ms and 10 ms (corresponding to a control loop or servo cycle frequency of between 2,000 to 100 Hz), though any suitable interval time period may be employed. Additional exemplary time periods are discussed further with reference to other embodiments described herein. An example of determining a power metric is discussed further with reference to the exemplary embodiments of FIGS. 4-6.

In some embodiments, an adaptive damper is implemented for a computer-assisted system, where the controller for damper is able to discard excessive negative energy generation over time to avoid delays in increasing damping in case of a change in environmental interaction of a manipulator arm. For example, if there is an excess amount of negative net energy generated then damping may be set to zero or approximately zero once the net energy reaches zero or is negative, and the damping may stay at zero or approximately zero until the net energy generated became positive again, a process which could take significant amount of time. By discarding the negative energy in some embodiments, the time to increase damping above zero or approximately zero may be reduced (e.g., damping may return more quickly than a control scheme that does not discard excessive negative energy). In some embodiments, the computer-assisted system increases the damping applied to the input device rapidly (e.g., more quickly than in embodiments without discarding excess negative energy) in response to a change in a power metric of a manipulator arm that may move into contact with a surface (e.g., a substantially rigid surface), bulk, or other material causing a rapid increase in the power metric. As discussed previously, in some embodiment, determine a damping applied to the input device may be based on a comparison (e.g., difference) between scaled power metrics for the input device and manipulator arm. In cases where the scaled power metric of the input device exceeds the scaled power metric of the manipulator arm (e.g., where the net power metric is negative), damping of the input device may be set to a baseline, minimum value. In some embodiments, the baseline, minimum value may be zero. In other embodiments, the baseline, minimum value may be non-zero. In some embodiments, a method of controlling a computer-assisted system includes decaying the excess input device power metric toward a net power metric of zero over time. That is, the method may include reducing an accumulation of a power metric of an instrument interaction with the passage of time, and may also include reducing an accumulation of a power metric of an input device interaction with the passage of time. For example, in some embodiments a constant linear slew rate may be employed to reduce the input device power metric until it is equal to the manipulator arm power metric. As another example, exponential decay may be employed to reduce the net power metric. Any suitable profile to reduce the input device power metric over time may be employed, as the present disclosure is not so limited. In this manner, when the manipulator arm power metric increases rapidly, damping may be applied to the input device without delay introduced by the excess input device power metric.

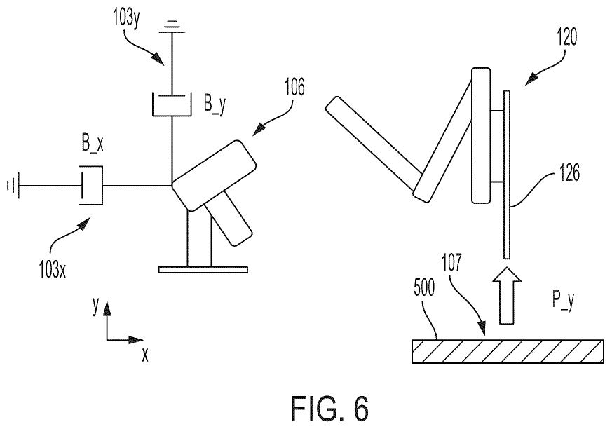

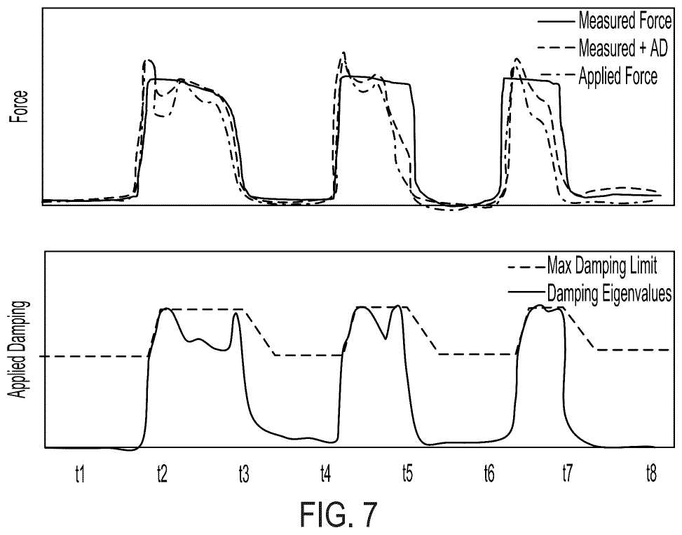

In addition to the above, in some embodiments, a damping limit is used to restrict the amount of damping applied to the input device. Applying higher damping to an input device may provide more stable force feedback control on the input device; however, applying higher damping may reduce the user-perceived quality of the feedback or responsiveness of the computer-assisted system. For example, movements of an input device while the input devices resists that motion or is slow relative to an input device with no damping may be undesirable in some applications of computer-assisted systems according to exemplary embodiments described herein (e.g., robotic surgery). Accordingly, in some instances, a damping limit is increased at a limited rate of change, thereby reducing the likelihood of a large increase in damping applied to the input device in a short span of time. A “short” span of time may be based on the forces, damping, and cycle times associated with the expected use of the computer-assisted system, so can vary from system to system. In some embodiments a “short” time span may include momentary increases (e.g., a step function). In some embodiments, a method of controlling a computer-assisted system may include limiting an amount of damping applied to the input device to a damping limit. In some embodiments, the method may include, in response to a determined amount of damping being greater than a damping threshold, increasing the damping limit at a known rate. In some embodiments, the damping threshold may be equivalent to the damping limit, and the damping threshold may also be variable based on the damping limit. For example, should the damping limit increase, the damping threshold may also correspondingly increase. In this manner, in some embodiments the damping limit may only increase when an amount of damping continually exceeds the increasing damping limit. The rate may be a monotonic rate (e.g., a positive monotonic rate), linear rate, exponential rate, or any other suitable rate profile. In some embodiments, the damping limit may be raised no further than a maximum damping limit, which may be predetermined based on the particular kinematics of an input device. For example, for a range of differences between an instrument power metric and an input device power metric, the damping may be non-proportional to the difference as damping is capped by the damping limit. In some embodiments, in response to the amount of damping being less than the damping limit, the damping limit may be decreased. For example, in some embodiments, the damping limit may decrease at monotonic, linear, or exponential rate. In some embodiments, the damping limit may not be decreased until a threshold period of time has passed. Such an arrangement may ensure the damping limit stays high in the case of a momentary decrease in an amount of damping applied to the input device. An exemplary implementation of damping limits and threshold is discussed further with reference to FIG. 7. According to exemplary embodiments described herein, a method may be described without specific reference to directionality. In some embodiment, power metrics and damping may be directional or assigned directionality. For example, power metrics and damping may be associated with three non-parallel translational degrees of freedom (e.g., x, y, and z directions). In such embodiments, power metrics and damping may be determined in each of the degrees of freedom. In some embodiments, damping and power metrics may be determined in rotational degrees of freedom (e.g., pitch, roll, and yaw directions). In some embodiments, power metrics and damping may be determined in any number of translational and rotational degrees of freedom or any sub-combination of rotational and translational degrees of freedom. In some embodiments, a power metric may be a scalar that is assigned directionality for the purposed of applying directional damping. An example of assigning directionality to a power metric is discussed further with reference to FIG. 6.

According to exemplary embodiments described herein, “damping” is a force that is opposed a velocity of an input device. The damping may be a resistive force that is proportional to the velocity of the input device. Accordingly, an increase in velocity of an input device may result in a corresponding increase in a damping force applied opposing the velocity of the input device. According to exemplary embodiments described herein, a method of controlling a computer-assisted system includes applying damping to an input device with a damping system. The damping may be applied via active or passive forces. For example, in some embodiments, actuators of an actuator system may be driven to apply actively damped forces in the form of damping in a direction or opposite that of the input device motion. Such damping forces may be incorporated into an overall force output of an actuator system. As another example, in some embodiments, a damping system may include an adjustable physical damper (e.g., a brake system) that may apply damping forces to the input device. As yet another example, a physical damper maybe physically adjusted to change a damping rate of the physical damper. Accordingly, determining and applying an amount of damping to such an input device may include adjusting the physical damper to achieve the desired amount of damping. Any combination of physical dampers and actuators operating as dampers may be employed as a part of a damping system, as the present disclosure is not so limited. While in some embodiments herein damping is proportional (e.g., linear) to velocity, in other embodiments, damping may be nonlinear, piecewise linear, etc.

According to exemplary embodiments described herein, power metrics of a manipulator arm and input device may be employed to determine a damping to be applied to an input device. In some cases, a manipulator arm and input device may be scaled with different scaling, such that when directly compared, a power metric of one may be much greater than a power metric of the other. For example, a manipulator arm may move a distance on the order of 1 m, whereas an input device may move a distance on the order of 10 cm. Accordingly, when directly compared, the manipulator arm power metric may be an order of magnitude greater than the input device power metric. Thus, in some embodiments, power metrics of the manipulator arm and input device may be determined with appropriate scaling such that they may be appropriately compared. In some embodiments, such scaling may be based on a scaling factor of position tracking between the input device and the manipulator arm or a scaling factor on forces experienced at each side of the telerobot. Any suitable scaling factor for the power metrics may be employed, as the present disclosure is not so limited. In the exemplary embodiments described herein, the power metrics of manipulator arms and input devices are generally described such that an increase in a power metric corresponds to an increase in amount of power or energy, a decrease in the power metric corresponds to a decrease in amount of power or energy, and a greater power metric corresponds to larger amount of power or energy than a lesser or smaller power metric. It can be appreciated that mathematical manipulation that merely changes the numeric value of a power metric doesn't change the underlying amount of power associated with that numeric value. For example, in a system whose mathematical manipulation or calculations associate lesser or smaller (including negative, or more negative) numeric values with power metrics corresponding to larger amounts of power or energy, the lesser or smaller numeric values corresponds to greater power metrics.

According to exemplary embodiments described herein, a method of controlling a computer-assisted system includes determining and applying damping as a part of a control loop. In some embodiments, the control loop may operate at a frequency suitable to damp vibration in a target frequency band. Methods according to embodiments described herein may be employed to damp vibration of an input device less than or equal to 10 Hz. Correspondingly, the control loop may operate at a frequency greater than 100 Hz to 1000 Hz. In some embodiments, the control loop may operate at a frequency range of more than ten times faster than the upper limit of the target frequency range. In some embodiments, the speed of the control loop may determine an accumulation time period for power metrics determined during a control loop cycle. In some embodiments, a power metric may be accumulated (e.g., integrated) over a period of time, which may be broken into a summation of a plurality of interval time periods (e.g., where an interval time period is associated with a control loop cycle time or a servo cycle time). A control loop may operate at any other frequencies depending on the particular computer-assisted application, as the present disclosure is not so limited in this regard.

This disclosure describes various devices, elements, and portions of computer-assisted devices and elements in terms of their state in three-dimensional space. As used herein, the term “position” refers to the location of an element or a portion of an element. Position can be determined in any number of dimensions suitable for the system, such as in one-dimensional, two-dimensional space, or three-dimensional space (e.g., three degrees of translational freedom along cartesian x-, y-, and z-coordinates). As used herein, the term “orientation” refers to the rotational placement of an element or a portion of an element. Orientation can be determined in any number of dimensions suitable for the system, such as in one, two, three degrees of freedom (e.g., in three degrees of rotational freedom such as roll, pitch, and yaw, can be represented with angle-axis, rotation matrix, quaternion representation, and/or the like). As used herein, and for a device with repositionable arms and/or other repositionable structures, the term “proximal” refers to a direction toward the base of the computer-assisted device along the kinematic chain of the computer-assisted device and “distal” refers to a direction away from the base along the kinematic chain.

As used herein, the term “pose” refers to the multi-degree of freedom (DOF) spatial position and orientation of a coordinate system of interest attached to a rigid body. In general, a pose includes a pose variable for each of the DOFs in the pose. For example, a full 6-DOF pose for a rigid body in three-dimensional space would include 6 pose variables corresponding to the 3 positional DOFs (e.g., x, y, and z) and the 3 orientational DOFs (e.g., roll, pitch, and yaw). Meanwhile, a 3-DOF position-only pose for a rigid body would include only pose variables for the 3 positional DOFs. And, a 3-DOF orientation-only pose for a rigid body would include only pose variables for the 3 rotational DOFs. Further, a velocity of the pose captures the change in pose over time (e.g., a first derivative of the pose). For a full 6-DOF pose for a rigid body, the velocity would include 3 translational velocities and 3 rotational velocities (e.g., both angular velocities and translational velocities). Poses with other numbers of DOFs would have a corresponding number of velocities translational and/or rotational velocities. Other examples for a rigid body include two translational and one rotational DOF, two or three translational DOFs, two or three rotational DOFs.

Aspects of this disclosure are described in reference to computer-assisted systems and devices, which may include systems and devices that are teleoperated, remote-controlled, autonomous, semiautonomous, robotic, and/or the like. Further, aspects of this disclosure are described in terms of an implementation using a surgical system. Knowledgeable persons will understand, however, that inventive aspects disclosed herein may be embodied and implemented in various ways, including robotic and, if applicable, non-robotic embodiments and implementations. Implementations on surgical systems are merely exemplary and are not to be considered as limiting the scope of the inventive aspects disclosed herein. For example, techniques described with reference to surgical instruments and surgical methods may be used in other contexts. Thus, the instruments, systems, and methods described herein may be used for humans, animals, portions of human or animal anatomy, industrial systems, general robotic, or teleoperated systems. As further examples, the instruments, systems, and methods described herein may be used for non-medical purposes including industrial uses, general robotic uses, sensing or manipulating non-tissue work pieces, cosmetic improvements, imaging of human or animal anatomy, gathering data from human or animal anatomy, setting up or taking down systems, training medical or non-medical personnel, and/or the like. Additional example applications include use for procedures on tissue removed from human or animal anatomies (without return to a human or animal anatomy) and for procedures on human or animal cadavers. Further, these techniques can also be used for medical treatment or diagnosis procedures that include, or do not include, surgical aspects.

It should also be noted that while some embodiments described herein employ rigid linkages, the present disclosure is not so limited. Techniques and methods described herein may be appliable to flexible robotic systems. For example, methods described herein may be applicable to rigid link robotic systems, flexible catheter systems, or other flexible robotic systems.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

FIG. 1 is a simplified diagram of an example computer-assisted system, according to various embodiments. In some examples, the computer-assisted system is a teleoperated system 100. In medical examples, teleoperated system 100 can be a teleoperated medical system such as a surgical system. As shown, teleoperated system 100 includes a follower device 104 that may be teleoperated by being controlled by one or more leader input devices 106 configured to accepted external input, described in greater detail below. Systems that include a leader device and a follower device are referred to as leader-follower systems. As shown in FIG. 1 the input devices are a part of an input system that includes a workstation 102 (e.g., a console). In other embodiments, the input system can be in any appropriate form and may or may not include a workstation 102.

In the exemplary embodiment of FIG. 1, the workstation 102 includes one or more input devices 106 which are configured to be contacted and manipulated by a user 108. For example, the workstation 102 can comprise one or more input devices 106 configured for use by the hands, the head, or some other body part of user 108. Input devices 106 in this example are supported by workstation 102 and can be mechanically grounded. In some embodiments, an ergonomic support 110 (e.g., forearm rest) can be provided on which the user 108 can rest his or her forearms. In some examples, user 108 can perform tasks at a worksite during a procedure by commanding follower device 104 using input devices 106. In some embodiments, the workstation 102 may be physically located near the follower device 104. In other embodiments, the workstation 102 may be remotely located from the follower device 104 and may communicate with the follower device via one or more communication protocols over a local area network, wide area network, and/or via the internet.

As shown in FIG. 1, the workstation 102 includes a display unit 112. The display unit 112 can display images for viewing by user 108. In some embodiments, the display unit 112 can be moved in various degrees of freedom to accommodate the viewing position of user 108 and/or to optionally provide control functions as another input device. In the example of teleoperated system 100, displayed images can depict a worksite at which user 108 is performing various tasks by manipulating input devices 106 and/or display unit 112. In some examples, images displayed by display unit 112 can be received by workstation 102 from one or more imaging devices arranged at a worksite. In other examples, the images displayed by display unit 112 can be generated by display unit 112 (or by a different connected device or system), such as for virtual representations of tools, the worksite, or for user interface components.

According to the embodiment of FIG. 1, when using the workstation 102, user 108 can sit in a chair or other support in front of workstation 102, position his or her eyes in front of display unit 112, manipulate input devices 106, and rest his or her forearms on ergonomic support 110 as desired. In some embodiments, user 108 can stand at the workstation or assume other poses, and display unit 112 and input devices 106 can be adjusted in position (height, depth, etc.) to accommodate user 108.

In some embodiments, the one or more input devices 106 can be ungrounded (ungrounded input devices being not kinematically grounded, such as input devices held by the hands of user 108 without additional physical support). Such ungrounded input devices can be used in conjunction with display unit 112. In some embodiments, user 108 can use a display unit 112 positioned near the worksite, such that user 108 manually operates instruments at the worksite, such as a laparoscopic instrument in a surgical example, while viewing images displayed by display unit 112.

Teleoperated system 100 can also include follower device 104, which can be commanded by workstation 102. In a medical example, follower device 104 can be located near an operating table (e.g., a table, bed, or other support) on which a patient can be positioned. In some medical examples, the worksite is provided on an operating table, e.g., on or in a patient, simulated patient, or model, etc. (not shown). The follower device 104 shown includes a plurality of manipulator arms 120, each manipulator arm 120 configured to couple to an instrument assembly 122. An instrument assembly 122 can include, for example, an instrument 126. In various embodiments, one or more of instruments 126 can include an imaging device for capturing images (e.g., optical cameras, hyperspectral cameras, ultrasonic sensors, etc.). For example, one or more of instruments 126 could be an endoscope assembly that includes an imaging device, which can provide captured images of a portion of the worksite to be displayed via display unit 112. One or more instruments 126 can also include instruments configured to function in an operating environment, and physically interact with physical objects or characteristics of the operating environment (e.g., in a medical example, an operating environment may be a work space within or outside a patient). Examples of potential instruments 126 may include, and are not limited to, graspers, scalpels, staplers, imagers or other sensors, cautery instruments, suction irrigators, and scissors. Any suitable instrument may be employed with a manipulator arm 120, as the present disclosure is not so limited.

In some embodiments, the manipulator arms 120 and/or instrument assemblies 122 can be controlled to move and articulate instruments 126 in response to manipulation of input devices 106 by user 108, and in this way “follow” through teleoperation the input devices 106. This enables user 108 to perform tasks at the worksite using the manipulator arms 120 and/or instrument assemblies 122. Manipulator arms 120 and follower device 104 are examples of repositionable structures on which instruments such as manipulating instruments or and/or imaging instruments including imaging devices can be mounted. The repositionable structure(s) of a computer-assisted system comprise the repositionable structure system of the computer-assisted system. For a surgical example, the user 108 could direct follower manipulator arms 120 to move instruments 126 to perform surgical procedures at internal surgical sites through minimally invasive apertures or natural orifices. The manipulator arms 120 and/or instrument assemblies 122 may also provide feedback information that is rendered to the user 108 through the one or more input devices 106. For example, in some embodiments, the manipulator arms 120 and/or instrument assemblies 122 may provide force information which is employed to apply feedback forces at the one or more input devices 106.

As shown, a control system 140 is provided external to workstation 102 and communicates with workstation 102. In other embodiments, control system 140 can be provided in workstation 102 or in follower device 104. As user 108 moves input device(s) 106, sensed spatial information including sensed position and/or orientation information is provided to control system 140 based on the movement of input devices 106. Control system 140 can determine or provide control signals to follower device 104 to control the movement of manipulator arms 120, instrument assemblies 122, and/or instruments 126 based on the received information and user input. In one embodiment, control system 140 supports one or more wired communication protocols. (e.g., Ethernet, USB, and/or the like) and/or one or more wireless communication protocols (e.g., Bluetooth, IrDA, HomeRF, IEEE 1002.11, DECT, Wireless Telemetry, and/or the like). In some embodiments, the control system 140 may be configured to implement exemplary methods herein and determine feedback forces to be applied to one or more input devices 106.

Control system 140 can be implemented on one or more computing systems. One or more computing systems can be used to control follower device 104. In addition, one or more computing systems can be used to control components of workstation 102, such as movement of a display unit 112.

As shown, control system 140 includes a processor 150 and a memory 160 storing a control module 170. In some embodiments, control system 140 can include one or more processors, non-persistent storage (e.g., volatile memory, such as random access memory (RAM), cache memory), persistent storage (e.g., a hard disk, an optical drive such as a compact disk (CD) drive or digital versatile disk (DVD) drive, a flash memory, etc.), a communication interface (e.g., Bluetooth interface, infrared interface, network interface, optical interface, etc.), and numerous other elements and functionalities. In addition, functionality of control module 170 can be implemented in any technically feasible software and/or hardware.

Each of the one or more processors of control system 140 can be an integrated circuit for processing instructions. For example, the one or more processors can be one or more cores or micro-cores of a processor, a central processing unit (CPU), a microprocessor, a field-programmable gate array (FPGA), an application-specific integrated circuit (ASIC), a digital signal processor (DSP), a graphics processing unit (GPU), a tensor processing unit (TPU), and/or the like. Control system 140 can also include one or more input devices, such as a touchscreen, keyboard, mouse, microphone, touchpad, electronic pen, or any other type of input device.

A communication interface of control system 140 can include an integrated circuit for connecting the computing system to a network (not shown) (e.g., a local area network (LAN), a wide area network (WAN) such as the Internet, mobile network, or any other type of network) and/or to another device, such as another computing system.

Further, control system 140 can include one or more output devices, such as a display device (e.g., a liquid crystal display (LCD), a plasma display, touchscreen, organic LED display (OLED), projector, or other display device), a printer, a speaker, external storage, or any other output device. One or more of the output devices can be the same or different from the input device(s). Many different types of computing systems exist, and the aforementioned input and output device(s) can take other forms.

In some embodiments, control system 140 can be connected to or be a part of a network. The network can include multiple nodes. Control system 140 can be implemented on one node or on a group of nodes. By way of example, control system 140 can be implemented on a node of a distributed system that is connected to other nodes. By way of another example, control system 140 can be implemented on a distributed computing system having multiple nodes, where different functions and/or components of control system 140 can be located on a different node within the distributed computing system. Further, one or more elements of the aforementioned control system 140 can be located at a remote location and connected to the other elements over a network.

Software instructions in the form of computer readable program code to perform embodiments of the disclosure can be stored, in whole or in part, temporarily or permanently, on a non-transitory computer readable medium such as a CD, DVD, storage device, a diskette, a tape, flash memory, physical memory, or any other computer readable storage medium. Specifically, the software instructions can correspond to computer readable program code that, when executed by a processor(s) (e.g., processor 150), is configured to perform embodiments of any of the methods described herein.

FIG. 2 is block diagram of a computer-assisted system according to some embodiments. As shown in FIG. 2, the computer-assisted system includes an input device 200. The input device may be configured to receive external input from a user. For example, the input device provides a user interface by which a user may impart physical motions and forces to the user interface which are reflected in a following manipulator arm. That is, the manipulator arm may move in accordance with inputs received at the input device based on a predetermined scaling factor. As shown in FIG. 2, the input device includes actuator(s) 204. The actuator(s) 204 may be configured to apply forces to the input device to provide feedback to a user of the input device. For example, the actuator(s) may be configured to apply forces based on forces sensed at the manipulator arm 250. The actuator(s) may include one more motors, servos, stepper motors, brushless motors, pneumatic actuators, or any other suitable actuator. The actuators may be a part of an actuator system that includes the actuator(s) and/or one or more other actuators. Accordingly, while actuator(s) are shown as being a part of an input device in FIG. 2, in other embodiments actuators may be provided as a separate actuator system. As shown in FIG. 2, the input device may optionally include damper(s) 206. The dampers may be adjustable dampers such that the amount of damping applied to the input device is controlled by processor(s) (e.g., 302) according to methods described herein. In some embodiments, the dampers may include brakes, which when engaged may apply constant or variable braking forces to the input device. In some embodiments, the actuator(s) 204 may function as dampers (e.g., by applying resistive forces opposing movement of the input device), in which case physical dampers may not be employed. According to exemplary embodiments herein, the actuator(s) and damper(s) may constitute portions of a damping system, which may include components of the input device and other components. A damping system may be configured to apply damping forces to the input device. Someone components of a damping system (e.g., actuators) may be employed as part of both feedback system and a damping system (e.g., a force output of an actuator may include both damping forces and feedback forces).

As shown in FIG. 2, the input device 200 also includes optional sensor(s) 208. The sensors may include accelerometers, inertial measurement units, contact sensors, force sensors, rotary encoders, potentiometers, position sensors, velocity sensors, or other desirable sensors configured to provide processor(s) (e.g., 302) information regarding the input device and its environment. In some embodiments, the actuator(s) 204 may provide information to processor(s) regarding the forces applied to the input device as well as the position of the input device. For example, forces may be measured by converting motor torque and/or current to forces. As another example, positions may be measured by monitoring steps of a stepper motor or otherwise monitoring the velocity of a motor over time. According to such examples, additional sensors may not be employed in an input device. In some embodiments, the input device may include one processor(s) (not shown). The processor(s) may be configured to execute programming instructions that may allow the input device to communicate, sense user inputs, and provide feedback to a user based on the output at a manipulator arm 250.

According to the embodiment of FIG. 2, a manipulator arm 250 is configured to cooperate with the input device 200. For example, the manipulator arm 250 may be configured to follow user input at the input device 200. Like the input device 200, the manipulator arm includes actuator(s) 254. The actuator(s) 254 may be configured to apply forces to the manipulator arm to move the manipulator arm and apply forces to an instrument environment. Additionally, the actuator(s) may be employed to operate one or more instruments associated with the manipulator arm 250. The actuator(s) may be configured to move the manipulator arm based on movements of the input device 200. The actuator(s) 254 may include one more motors, servos, stepper motors, brushless motors, pneumatic actuators, or any other suitable actuator. As shown in FIG. 2, the manipulator arm may optionally include one or more instruments 256. The one or more instruments may be coupled to the manipulator arm, and in some embodiments may be removable and interchangeable. The instrument(s) 256 may include one or more of an imaging device, graspers, scalpels, staplers, knives, cautery instruments, and scissors.

As shown in FIG. 2, the manipulator arm 250 also includes optional sensor(s) 258. The sensors may include accelerometers, inertial measurement units, contact sensors, force sensors, rotary encoders, potentiometers, position sensors, velocity sensors, or other desirable sensors configured to provide processor(s) (e.g., 302) information regarding the manipulator arm and its environment. In some embodiments, the sensor(s) may be configured to provide information to processor(s) (e.g., 302) regarding contact forces on the manipulator arm, position of the manipulator arm, and velocity of the manipulator arm. In some embodiments, the actuator(s) 254 may provide information to processor(s) (e.g., 302) regarding the forces applied to the manipulator arm as well as the position of the manipulator arm. For example, forces may be measured by converting motor torque and/or current to forces. As another example, positions/and velocities may be measured by monitoring steps of a stepper motor or otherwise monitoring the velocity of a motor over time. In some embodiments, the sensor(s) 258 may include force sensors located at a distal portion of an instrument. In some embodiments, a distal portion of the instrument comprises an end effector of the instrument. The end effector is configured to interact with the environment, such as to manipulate and/or sense data from the environment. Examples of end effectors include those with manipulating components such as fingers, suction devices, irrigators, cutters. Examples of end effectors also include those with sensor components such as optical imaging devices, temperature or force sensors, and ultrasonic probes. In some embodiments, the sensor(s) 258 may include one or more strain gauges positioned on a portion of the manipulator arm. A manipulator arm 250 may include any suitable number and type of sensors, as the present disclosure is not so limited. In some embodiments, the manipulator arm may include one processor(s) (not shown). The processor(s) may be configured to execute programming instructions that may allow the manipulator arm to communicate, sense its environment, and interact with its environment based on the input at the input device 200.

According to some embodiments as shown in FIG. 2, the computer-assisted system includes a control system 300 which may coordinate and control interoperation between the input device 200 and the manipulator arm 250. The control system 300 may implement methods according to exemplary embodiments described herein for controlling the operation of the input device 200 and the manipulator arm 250. In particular, the control system receives input from the input device 200 and may provide feedback to the input device from the manipulator arm 250. The control system also sends commands to the manipulator arm 250 based on the input received at the input device 200 and receives sensor feedback from the manipulator arm. system 300. The control system 300 may control the output of the actuator(s) of both the manipulator arm 250 and the input device 200 in one or more control loops. In some embodiments, the control system 300 may cooperate with the processor(s) of the input device and manipulator arm to control the input device and manipulator arm.

As shown in FIG. 2, the control system includes one or more processors 302 which are configured to execute programming instructions that cause the one or more processors to perform exemplary methods described herein. The programming instructions may be stored on memory 304, which may be non-transitory memory. In some embodiments. the control system 300 may comprise, or communicate with, any processor(s) of the input device and/or any processors of the manipulator arm, to control the input device and manipulator arm. In some embodiments, the control system may physically be implemented in one electronic circuit (e.g., one integrated circuit). In other embodiments, the control system may be physically implemented in multiple electronic circuits (e.g., multiple integrated circuits) physically co-located or distributed relative to each other. In some embodiments, a processor of the control system may be physically disposed in or on the input device, the manipulator arm, an auxiliary tower, or a user console, another part of the computer-assisted system, or any combination thereof. In the examples described herein, the control system 300 also includes a communications interface 306 which may facilitate communication of commands, requests, and information between the manipulator arm, input device, and control system 300. The communications interface 306 can include a circuit for connecting the control system to a network (not shown) (e.g., a local area network (LAN), a wide area network (WAN) such as the Internet, mobile network, or any other type of network) and/or to another device, such as another control system. In some embodiments, the manipulator arm 250 and input device 200 may also include communications interfaces to facilitate communication with the control system 300.

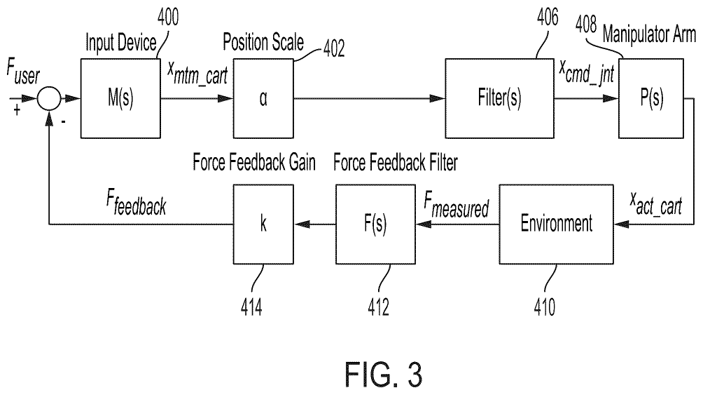

FIG. 3 is a diagram of a control loop for a computer-assisted system according to some embodiments. As shown in FIG. 3, input to the control loop is force from a user. The force from a user may be applied to an input device and may be monitored by one or more processors of the input device. That is, the one or more processors of the input device may receive information from one or more sensors and/or actuators of the input device which may be employed to determine a force input from the user. In block 400, the overall motion and force of the input device is represented. The motion and force are a result of a combination of the force input by the user and feedback from the control loop. In block 402, the position movements and/or forces of the input device are scaled. The scale may be based on kinematic differences between the input device and a manipulator arm. For example, the input device may have a range of motion much smaller than that of a manipulator arm, such that the motions and forces of the input device are scaled to match the larger range of motion of the manipulator arm. In some embodiments, the scaled motions and forces are employed to as to command a virtual manipulator arm, as the information is transferred from the physical input device to the physical manipulator arm. Such a step may employ inverse kinematics to provide commanded positions or motions to a virtual or actual manipulator arm. Alternatively, such a step may not employ inverse kinematics. For example, some systems utilize a user input device for commanding a manipulator arm with similar or identical kinematics to the input device, and such systems may command the joint positions or motions of the manipulator arm directly based on the joints positions or motions of the input device, without employing inverse kinematics. In block 406, filters may be employed to reduce noise or adjust phase. Any suitable filter may be employed, as the present disclosure is not so limited. In block 408, the manipulator arm is controlled based on the filtered signal provided by the virtual manipulator arm. That is, the manipulator arm is moved proportionally based on the movement at the input device in block 400.

As shown in FIG. 3, the manipulator arm of block 408 provides a feedback signal. The feedback signal may be based on forces measured at the manipulator arm, which may be rendered to the input device so that the user feels feedback in response to forces experienced by the manipulator arm. In particular, the manipulator arm may interact with its environment in block 410. As a result of this interaction, forces may be measured using one or more sensors onboard the manipulator arm. This force signal may be filtered in block 412 to reduce noise or target specific frequency response ranges. The force feedback filter of block 412 may include the determination and application of damping to the input device to avoid instabilities in the input device. Accordingly, block 412 may be representative of exemplary methods described herein. In other embodiments, a separate branch of the loop may extend from block 400, which may provide damping input to the input device output. For example, an additional loop may apply force based on a determined damping and a velocity of the input device. In block 414, a force feedback gain may be applied to the filtered force feedback signal, to alter the amount of feedback force rendered to the user at the input device. As shown in FIG. 3, the force feedback is then rendered back into the input device thereby forming the closed control loop. As will be discussed further blow, the control loop shown in FIG. 3 may include determining and applying damping to the input device.

FIG. 4 is a simplified diagram of a computer-assisted system during operation according to some embodiments demonstrating a method of controlling the computer-assisted system. As shown in FIG. 4, the computer-assisted system includes an input device 106 and a manipulator arm 120. The manipulator arm includes an instrument 126 having a distal portion 107 (e.g., an end effector, etc.) which physically interacts with an environment 500. The environment may include different objects with different mechanical characteristics (e.g., tables, working surfaces, components, floor, wall, ceiling) and/or different non-solid materials (e.g., gels, liquids, gases), depending on the particular application. Examples of objects include subjects (e.g., patients, parts of patient anatomy, industrial work pieces, training devices). As discussed previously, the input device 106 and the manipulator arm 120 may be remotely located from each other and may communicate via a control system configured to coordinate the operation of the input device and manipulator arm. The manipulator arm may be configured to proportionally follow movements and forces applied by a user to the input device. That is, user input received at the input device may be the basis for commands sent to the manipulator arm to move and interact with the environment 500.

According to the embodiment of FIG. 4, the parameters employed in determining a level of damping for the input device are shown. In particular, according to the embodiment of FIG. 4, a damping of the input device 106 is determined based on power metrics associated with the input device and the manipulator arm 120. As shown in FIG. 4, a power metric between the manipulator arm and environment may be determined as P_env. This power may be calculated by determining a force and velocity of the portion manipulator arm engaged with the environment 500. For example, the power metric may be calculated as a force at the distal portion 107 times a velocity of the distal portion. In some embodiments, a power metric may be determined by a multiple of a rotational velocity and a torque applied. In some embodiments, a power metric may be determined by a multiple of a translational velocity and a translational force as in FIG. 4. As shown in FIG. 4, a power metric of the input device may be determined as a combination of the power of the force feedback P_ffb and the power of the damper P_Damper. In some embodiments, these power metrics may be determined as a function of a velocity of the input device 106 times the forces applied to the input device with one or more actuators. Based on a combination of these power metrics, a damping of the input device 106 may be determined. In particular, in some embodiments, an excess power may be determined based on a comparison of the power metrics according to the following equation:

$$P\_excess = P\_ffb - α P\_Damper - P\_env$$

where α is a scaling factor. In some embodiments, if P_excess is less than or equal to 0, the damping of the input device may be set to zero. In some embodiments, if P_excess is greater than 0, damping may be set based on the following equation:

$$B\_A . Damper = K * P\_Excess$$

where K is a gain factor that may be set depending on the particular kinematics of the computer-assisted system. In some embodiments as discussed previously, an accumulation of the power metrics may be employed to determine a damping of an input device. For example, an excess energy, E_Excess may be determined by taking the integral of the P_Excess over a particular time period. In such an embodiment, the energy may be employed to set the damping factor where the energy is greater than zero according to the following equation:

$$B\_A . Damper = K * E\_Excess$$

where K is a gain factor that may be set depending on the particular kinematics of the computer-assisted system. Accordingly, damping for the input device may be determined and applied to the input device according to power measurements at the input device and the manipulator arm.

FIG. 5 is a simplified diagram of an input device 106 of a computer-assisted system according to some embodiments. As shown in FIG. 5, the input device 106 includes a virtual damper 103. The virtual damper 103 has a damping coefficient B_A.Damper, which may be determined according to the process described above with reference to FIG. 4. according to some embodiments. In the embodiment of FIG. 5, the damper may be virtual, in that actuators are configured to apply active forces in a direction opposing a direction of velocity of the input device. In other embodiments, the damper may be physical damper with an adjustable damping rate. While in the embodiment of FIG. 5 a damper in one direction is shown, in other embodiments damping may be applied in multiple degrees of freedom, including translation degrees of freedom (e.g., x, y, and z cartesian directions), rotational degrees of freedom (e.g., pitch, roll, and yaw directions), or any combination or sub-combination thereof. Applying damping in multiple directions will be described further with reference to FIG. 6.

FIG. 6 is a simplified diagram of a computer-assisted system during operation according to some embodiments. In particular, FIG. 6 shows the use of directionality in determining and applying damping to an input device 106. Like the embodiment of FIG. 5. the computer-assisted system of FIG. 6 includes a manipulator arm 120. The manipulator arm includes an instrument 126 having a distal portion 107 (e.g., an end effector) which physically interacts with an environment 500. The environment may include objects with different mechanical characteristics and/or different non-solid materials, similar to described in conjunction with the embodiment of FIG. 4, depending on the particular application.

According to the embodiment of FIG. 6, two Cartesian directions are shown representing two translational degrees of freedom of the input device 106 and the manipulator arm 120. That is, both the input device 106 and the manipulator arm 120 may be able to move in the x and y directions as shown in FIG. 6. In some embodiments, the reference frame for cartesian directions may be different between the input device 106 and the manipulator arm 120. That is, in a global reference frame the x direction of the input device may not be parallel with the x direction of the manipulator arm. Rather, movements of the input device in the x-direction may be transformed to movements of the manipulator arm in the x direction based on the local reference frame of the manipular arm. In some embodiments the cartesian directions of the input device may be parallel to the cartesian directions of the manipulator arm in a global reference frame, as the present disclosure is not so limited.

According to the embodiment of FIG. 6, power metrics of the input device 106 and the manipulator arm 120 are determined in three non-parallel translational degrees of freedom (e.g., x, y, and z directions). In other embodiments power may also be determined in one or more rotational degrees of freedom, or any subset of translational and rotational degrees of freedom, as the present disclosure is not so limited. As discussed previously, a power metric may be determined as a product of a force measurement and a velocity measurement. In some embodiments, an instrument power metric may be determined by projecting the force applied to the input device onto the translational degrees of freedom (or other degrees of freedom) of the input device. In some embodiments, such a projection may be accomplished by employing a dot product. The projected force may then be multiplied by the input device velocity in each respective direction to determine an input device power metric matrix. Similarly, an instrument power metric may be determined by projecting force measured at the instrument onto the translational degrees of freedom (or other degrees of freedom) of the instrument. The projected instrument forces may then be multiplied by the instrument velocities in each respective direction to determine an instrument power metric matrix. A net power generation may then be obtained by comparing the input device power matrix and the instrument power matrix as described with reference to FIGS. 4-5, which may be employed to determine a damping of the input device. The multiple values in the matrix may correspond to the different degrees of freedom the input device that are damped. In effect, this process may allow determination of a damping coefficient of virtual dampers each corresponding to a degree of freedom of the input device. In this manner, damping may only be applied in directions in which the power comparison yields excess power. According to such a method, damping can be applied in an anisotropic manner, where damping differs along the various degrees of freedom based on the magnitude of the excess power along each respective degree of freedom. The damping may be applied in only direction resting movement of the user input device as a user operates the input device.

The axes shown in FIG. 6 are exemplary, and in some embodiments may change as an instrument environmental interaction or input device environmental interaction changes. For example, in the FIG. 6 example, if the environmental contact direction were rotated 45 degrees about an axis coming directly out of the page, the axes for the purposes of determining power metrics and damping may also be rotated 45 degrees about the same axis. Accordingly, a damping coordinate frame may be based on a direction in which forces are applied between an instrument and an instrument environment and an input device and an input device environment.

FIG. 6 depicts an exemplary instrument power metric and associated virtual dampers corresponding to the x and y translational degrees of freedom. The input device 106 may have one or more additional degrees of freedom, but only the x and y translation degrees of freedom are shown for simplicity of explanation. The right side of FIG. 6 shows the manipulator “follower” portion. As shown in the rights side of FIG. 6, the distal portion 107 of the instrument 126 is pressed against the environment 500, which may be a surface perpendicular to the y direction, with a force in the negative-y direction, which is opposite the direction represented by arrow P_y. Accordingly, as shown in FIG. 6, an instrument power metric may reflect the force and velocity in the y direction, represented by the arrow P_y. However, for simplicity of explanation and based on the force applied by and movement of the instrument 126 being directly along the direction represented by arrow P_y, there is not force applied to the instrument in the X direction. In this example, a power metric in the x direction would be a value effectively associated with no power or energy (e.g., “zero”, or some other baseline value) and is not shown in FIG. 6. The left side of FIG. 6 shows the input device “leader” portion. On the input device side, an x direction damper 103x and a y direction damper 103y are shown. These dampers may be virtual dampers and are representative of passive or active forces applied to the input device based on a level of damping determined according to exemplary methods described herein. For simplicity of explanation, damper 103x and damper 103y are described here as modelled by pure viscous damping, and in other embodiments dampers with more complex or nonlinear damping responses can be used. In the FIG. 6 example, the x direction damper 103 has a coefficient of damping B_x and the y direction damper 103y has a coefficient of damping B_y. In the example shown in FIG. 6, the instrument power metric P_y may be “non-zero”, and effectively indicate amount of power or energy that is different from no power or no energy (for example, the power metric P_y may be zero, or a baseline value) The non-zero power metric P_y and may cause excess energy which is employed to determine the y direction coefficient of damping B_y. For example, B_y may be set as being proportional to a difference in the instrument power metric P_y and a corresponding y direction input device power metric. Accordingly, the input device 106 may be damped in the y direction. In contrast, because the instrument power metric in the x direction is zero or otherwise less than or equal to an x direction input device power metric, the x direction coefficient of damping B_x may be set to zero or another baseline value. In this manner, damping may be applied to the input device in the direction in which excess power is being generated, so as to avoid instabilities in force feedback in those directions, while retaining little to no damping in other stable directions without excess power so as to not impede user feel in those directions.

In some embodiments as discussed preciously, a power metric matrix may be integrated or otherwise accumulated over time to obtain an energy matrix. In some embodiments, such an energy matrix may be employed for determination of multiple damping coefficients associated with each degree of freedom of an input device. In some embodiments, the damping may be determined as a function of a gain value time as a net energy matrix. Accordingly, methods described herein with reference to one or more power matrices may be employed using an energy calculation instead.

It should be noted that while two translational degrees of freedom (x and y) are shown and described with reference to FIG. 6 in a planar example, in other embodiments any suitable degrees of freedom may be employed, including any combination of translation and/or rotational degrees of freedom. For example, in some embodiments power metrics and damping may be determined in rotational degrees of freedom only rather than translational degrees of freedom as shown in FIG. 6.

FIG. 7 depicts graphs of experimental results of a computer-assisted system according to some exemplary embodiments described herein. In particular, the data shown in FIG. 7 is representative of an instrument moving into and out of contact with a surface in its environment. In particular, the instrument is moved into contact and out of contact successively in one direction. The data shown in FIG. 7 is representative of a single direction for determination of force, power metrics, and damping, though the data in FIG. 7 may be extended to multiple directions corresponding to multiple degrees of freedom.

As discussed previously, in some embodiments, a method of controlling a computer-assisted system includes limiting the amount of damping applied to the input device. FIG. 7 depicts results of a method implementing such a technique to improve user performance feel while allowing damping to increase in unstable situations. In some embodiments, determining an amount of damping to apply to an unput device may include limiting the amount of damping to a damping limit. such a damping limit is shown in the lower graph. In some embodiments, the damping limit may be static, whereas in other embodiments, the damping limit may adjust to accommodate additional damping when appropriate. In some such embodiments, the damping limit may be configured to increase by a certain rate when the amount of damping determined exceeds a damping threshold. In some cases, the damping threshold and damping limit may be the same, such that determination of an amount of damping greater than the imposed damping limit causes the damping limit to increase at the rate. The rate may be a maximum rate, configured to limit the speed at which the damping limit increases, thereby eliminating rapid spikes in damping of the input device. In some embodiments, the damping limit may increase proportionally with the determined amount of damping. An example of the damping limit increasing is shown in FIG. 7. In the depicted embodiment, the rate limiting the increase in damping is a linear slew rate. As the damping limit increases, so may the amount of damping applied to the input device. In some embodiments, the damping limit may be capped at a maximum damping limit, which may be determined based on the particular kinematics and applications of the computer-assisted system.

In some embodiments as shown in FIG. 7, a damping limit may also return to its original value when the additional damping is not desirable. In some embodiments, the damping limit may be lowered when the amount of damping is less than the damping limit for a threshold period of time. In the example of FIG. 7, this is shown as the damping limit decreases when the damping eigenvalues fall below the limit. The threshold period of time may be any suitable period of time, including period between 0 and 1 second, 0.01 second and 0.1 seconds, 0.1 and 0.5 seconds, or any other suitable range. In some embodiments, no time period may be employed, and the damping limit may be lowered once the determined amount of damping is less than the damping limit. In some embodiments as shown in FIG. 7, the damping limit may be reduced at another rate. That is, the damping limit may be configured to decrease at a capped rate, such that the damping limit does not immediately return to the original value. Such an arrangement may be beneficial to provide stability during time periods where there may be multiple interactions in quick succession between and instrument and its environment. In the embodiment of FIG. 7, the rate of decrease is a linear slew rate, though other arrangements are contemplated, including exponential decay or other monotonically decreasing rates.

FIG. 8 depicts a flow chart for a method of operating a computer-assisted system according to some embodiments. In block 600, an instrument power metric indicative of an amount of power of an instrument interaction is determined. The instrument interaction comprises a physical interaction between at least a portion of the instrument (e.g., a distal portion of the instrument such as an end effector of the instrument) and an instrument environment containing the instrument. In some embodiments, the instrument interaction may include interactions with tissues or other objects, or non-solid materials, such as with the surfaces or bulks of such objects or solid materials. As discussed previously, in some embodiments the power metric may be determined with respect to multiple degrees of freedom, including any subset of translational and rotational degrees of freedom of the instrument. As discussed previously, the instrument power metric may be representative of the energy of the interaction of the instrument with the instrument environment (e.g., instantaneous power measured over a time period, an amount of energy applied or received over the time period). In block 602, an input device power metric indicative of an amount of power of a feedback provided by the input device in response to the instrument interaction is determined. In some embodiments the input device power metric may be determined in multiple degrees of freedom, including any subset of translational and rotational degrees of freedom of the input device. The input device power metric may be determined, for example, as described in reference to FIG. 4.

As shown in FIG. 8, in block 604 a damping to be applied to the input device is determined based on the instrument power metric and the input device power metric. For example, in comes embodiments, the input device power metric and instrument power metric are compared. Such a comparison may yield an excess power metric generated by a control loop for the computer-assisted system (e.g., a net energy comparison between the input device environmental interaction and an instrument environmental interaction). This excess power metric may be employed to determine the damping. In some embodiments, the damping may be proportional to the excess power. As discussed previously, the power metrics may be representative of energy (e.g., power over a period of time), and, in some embodiments, power may be integrated or summed over time to determine the power metrics, which may be compared and employed to determine the damping applied by a damping system. In block 606, a damping system caused to apply the determined damping to the input device. The damping system may include a physical damper such as a brake, which may be adjusted to apply the determined damping, in some embodiments. In other embodiments, the damping system may include or cooperated with an actuator system to apply active forces resisting motion of the input device, in accordance with the determined damping. The applied damping in such an arrangement may be incorporated into a total force output commanded to a particular actuator. That is, the application of damping may be incorporated into a total output force of an actuator, in some embodiments. As shown in FIG. 8, the method may be repeated as a part of a control loop for the computer assisted system.

The above-described embodiments of the technology described herein can be implemented in any of numerous ways. For example, the embodiments may be implemented using hardware, software or a combination thereof. When implemented in software, the software code can be executed on any suitable processor or collection of processors, whether provided in a single computer or distributed among multiple computers. Such processors may be implemented as integrated circuits, with one or more processors in an integrated circuit component, including commercially available integrated circuit components known in the art by names such as CPU chips, GPU chips, microprocessor, microcontroller, or co-processor. Alternatively, a processor may be implemented in custom circuitry, such as an ASIC, or semicustom circuitry resulting from configuring a programmable logic device. As yet a further alternative, a processor may be a portion of a larger circuit or semiconductor device, whether commercially available, semi-custom or custom. As a specific example, some commercially available microprocessors have multiple cores such that one or a subset of those cores may constitute a processor. Though, a processor may be implemented using circuitry in any suitable format.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, the embodiments described herein may be embodied as a computer readable storage medium (or multiple computer readable media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other tangible computer storage medium) encoded with one or more programs that, when executed on one or more computers or other processors, perform methods that implement the various embodiments discussed above. As is apparent from the foregoing examples, a computer readable storage medium may retain information for a sufficient time to provide computer-executable instructions in a non-transitory form. Such a computer readable storage medium or media can be transportable, such that the program or programs stored thereon can be loaded onto one or more different computers or other processors to implement various aspects of the present disclosure as discussed above. As used herein, the term “computer-readable storage medium” encompasses only a non-transitory computer-readable medium that can be considered to be a manufacture (i.e., article of manufacture) or a machine. Alternatively or additionally, the disclosure may be embodied as a computer readable medium other than a computer-readable storage medium, such as a propagating signal.

The terms “program” or “software” are used herein in a generic sense to refer to any type of computer code or set of computer-executable instructions that can be employed to program a computer or other processor to implement various aspects of the present disclosure as discussed above. Additionally, it should be appreciated that according to one aspect of this embodiment, one or more computer programs that when executed perform methods of the present disclosure need not reside on a single computer or processor but may be distributed in a modular fashion amongst a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that conveys relationship between the fields. However, any suitable mechanism may be used to establish a relationship between information in fields of a data structure, including through the use of pointers, tags or other mechanisms that establish relationship between data elements.

Various aspects of the present disclosure may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the embodiments described herein may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Further, some actions are described as taken by a “user.” It should be appreciated that a “user” need not be a single individual, and that in some embodiments, actions attributable to a “user” may be performed by a team of individuals and/or an individual in combination with computer-assisted tools or other mechanisms.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

1. A computer-assisted system comprising:
a manipulator arm;
an input device configured to accept user commands to move the manipulator arm;
a damping system coupled to the input device; and
a controller comprising at least one processor, the controller configured to:
determine an instrument power metric indicative of an amount of power of an instrument interaction, the instrument interaction comprising a physical interaction between at least a portion of an instrument and an instrument environment containing the instrument, the instrument supported by the manipulator arm,
determine an input device power metric indicative of an amount of power of a feedback provided by the input device in response to the instrument interaction, determine a damping to be applied to the input device based on the instrument power metric and the input device power metric, and
cause the damping system to apply the damping to the input device.

2. The computer-assisted system of claim 1, wherein the portion of the instrument comprises a distal portion of the instrument.

3. The computer-assisted system of claim 1, wherein the controller is configured to determine the damping by:
in response to the input device power metric being greater than or equal to the instrument power metric, setting the amount of damping to a minimum damping.

4. The computer-assisted system of claim 3, wherein the controller is configured to determine the damping further by:
in response to the input device power metric being less than the instrument power metric, setting the amount of damping to a higher damping greater than the minimum damping.

5. The computer-assisted system of claim 1, wherein when the instrument power metric is greater than the input device power metric, an amount of the damping to be applied to the input device has a positive monotonic relationship with an amount by which the instrument power metric is greater than the input device power metric.

6. The computer-assisted system of claim 5, wherein the positive monotonic relationship is a strictly monotonic relationship.

7. The computer-assisted system of claim 5, wherein the amount by which the instrument power metric is greater than the input device power metric comprises an amount of the instrument power metric minus an amount of the input device power metric.

8. The computer-assisted system of claim 1, wherein:
the controller is configured to determine the damping by:
determining a damping coefficient based on a difference between the instrument power metric and the input device power metric.

9. The computer-assisted system of claim 8, wherein the damping coefficient is proportional to the difference for a first range of differences where the instrument power metric is greater than the input device power metric.

10. The computer-assisted system of claim 9, wherein:
the damping coefficient is not proportional to the difference for a second range of differences where the instrument power metric is greater than the input device power metric, the second range different from the first range; or
the damping coefficient is not proportional to the difference for a third range of differences where the instrument power metric is not greater than the input device power metric.

11. The computer-assisted system of claim 1,
wherein the controller is configured to determine the damping by:
limiting the amount of the damping to a damping limit.

12. The computer-assisted system of claim 11, wherein the controller is further configured to:
in response a determination that an amount of the damping is greater than a damping threshold, increasing the damping limit at a first rate.

13. The computer-assisted system of claim 12, wherein the controller is further configured to:
limit the damping limit to no more than a maximum damping limit.

14. The computer-assisted system of claim 12, wherein the controller is further configured to:
in response to the amount of the damping being less than the damping limit for a threshold period of time, decrease the damping limit.

15. The computer-assisted system of claim 1,
wherein the controller is configured to cause the damping system to apply the damping to the input device by:
causing a physical adjustment of an adjustable damper of the damping system;
driving an actuator system to apply actively damped force or torque to the input device; or
causing a brake system of the damping system to apply braking forces to the input device.

16. The computer-assisted system of any of claim 1,
wherein the controller is configured to cause the damping system to apply the damping to the input device by:
driving an actuator system to apply actively damped force or torque to the input device in a direction opposite to a direction of motion of the input device.

17. The computer-assisted system of any of claim 1,wherein:
the controller is configured to determine the instrument power metric by determining an accumulation of a power of the instrument interaction over a time period; and
the controller is configured to determine the input device power metric by determining an accumulation of a power of the input device interaction over the time period.

18. The computer-assisted system of claim 17, wherein:
the controller is configured to determine the instrument power metric further by reducing the accumulation of the power of the instrument interaction with a passage of time; and
the controller is configured to determine the input device power metric further by reducing the accumulation of the power of the input device interaction with the passage of time.

19. The computer-assisted system of claim 1,
wherein:
the controller is configured to determine the instrument power metric by: determining at least one multiple selected from the group consisting of:
a multiple of a translational velocity of the instrument along an instrument translational degree of freedom and a force applied by the instrument to the instrument environment in the instrument translational degree of freedom, and
a multiple of a rotational velocity of the instrument along an instrument rotational degree of freedom and a torque applied by the instrument to the instrument environment in the instrument rotational degree of freedom; and
the controller is configured to determine the input device power metric by: determining at least one multiple selected from the group consisting of:
a multiple of a translational velocity of the input device in an input device translational degree of freedom and a force applied by the input device to the input device environment in the input device translational degree of freedom, and a multiple of a rotational velocity of the input device in an input device rotational degree of freedom and a torque applied by the input device to the input device environment in the input device rotational degree of freedom.

20. The computer-assisted system of claim 1,
wherein:
the computer-assisted system is configured to move the instrument in a plurality of instrument degrees of freedom;
the input device is configured to move in a plurality of input device degrees of freedom, each degree of freedom of the plurality of input device degrees of freedom corresponding to a degree of freedom of the plurality of instrument degrees of freedom;
the controller is configured to determine the instrument power metric by for each degree of freedom of a plurality of non-parallel instrument degrees of freedom, calculating an instrument power component based on a multiple of instrument velocity and instrument force in that instrument degree of freedom;
the controller is configured to determine the input device power metric by for each degree of freedom of a plurality of input device degrees of freedom, calculating an input device power component based on a multiple of input device velocity and input device force in that input device degree of freedom; and
the controller is configured to determine the damping by for each degree of freedom of the plurality of input device degrees of freedom, determining a damping amount in that input device degree of freedom based on the input device power component along that input device degree of freedom and the instrument power component along the degree of freedom corresponding to that input device degree of freedom.

21. The computer-assisted system of claim 20, wherein:
the plurality of instrument degrees of freedom comprises a first instrument translational degree of freedom, a second instrument translational degree of freedom, and a third instrument translational degree of freedom; and
the plurality of input device degrees of freedom comprises a first input device translational degree of freedom associated with the first instrument translational degree of freedom, a second input device translational degree of freedom associated with the second instrument translational degree of freedom, and a third input device translational degree of freedom associated with the third instrument translational degree of freedom.

22. A method of controlling a computer-assisted system including a manipulator arm, an input device configured to accept user commands to move an instrument, and a damping system coupled to the input device, the method comprising:
determining an instrument power metric indicative of an amount of power of an instrument interaction, the instrument interaction comprising a physical interaction between at least a portion of an instrument and an instrument environment containing the instrument, the instrument supported by the manipulator arm;
determining an input device power metric indicative of an amount of power of a feedback provided by the input device in response to the instrument interaction;
determining a damping to be applied to the input device based on the instrument power metric and the input device power metric; and
causing the damping system to apply the damping to the input device.

23-34. (Canceled)

35. At least one non-transitory computer-readable storage medium storing programming instructions that, when executed by at least one processor associated with a computer-assisted system, causes the at least one processor to perform the operations,
wherein the computer-assisted system comprises:
a manipulator arm;
an input device configured to accept user commands to move the manipulator arm; and
a damping system coupled to the input device;
wherein the operations comprise:
determining an instrument power metric indicative of an amount of power of an instrument interaction, the instrument interaction comprising a physical interaction between at least a portion of an instrument and an instrument environment containing the instrument, the instrument supported by the manipulator arm;
determining an input device power metric indicative of an amount of power of a feedback provided by the input device in response to the instrument interaction;
determining a damping to be applied to the input device based on the instrument power metric and the input device power metric; and
causing the damping system to apply the damping to the input device.
